(19) United States
(12) Patent Application Publication
(10) Pub. No.: US 2008/0047041 A1
(43) Pub. Date: Feb. 28, 2008

(54) SIMPLE SUN VISOR

(76) Inventor: Ka Ling Daniel Si, Hong Kong (HK)

Correspondence Address: ERIC CHAN, 42 PIN OAKS DRIVE, PHOENIXVILLE, PA, 19460, US

(21) Appl. No.: 11/842,943

(22) Filed: Aug. 21, 2007

(30) Foreign Application Priority Data
Aug. 22, 2006 (HK) 06109331.3

(51) Int. Cl. A61F 9/06

(52) U.S. Cl. 2/12

(57) ABSTRACT

The present invention provides a simple sun visor which comprises a body and a visor. The body comprises two elastic thin supporting arms. The two supporting arms form a certain curvature after formation and engage with the two ends of the visor respectively. The two supporting arms and the visor are engaged by means of hinges, insertion, clips or fixation. The upper edge of the visor is disposed with a soft elastic rim having waterproof, slip-proof and shockproof properties. The present invention uses fixing construction of thin supporting arms which is similar to that of the arms of eyeglasses. The present invention is compact in construction and lighter than visors of the same size available in the marketplace. If the present invention is disposed with foldable, sliding or rotational supporting arms, it is more convenient to fold and unfold. The present invention is floatable on water and easy to clean and dry. It is simple to use, small in size, comfortable to wear, low in production cost and highly flexible in design. It is easy to store or carry around when not in use and is extremely suitable for traveling, going outdoors, and playing all kinds of sports.

### BACKGROUND OF THE INVENTION

The present invention relates to a sun visor and more particularly pertains to a simple sun visor.

During outdoor activities, many people choose to wear sun caps to shield themselves from the sun. A sun cap generally comprises a cap body and a visor. When a user wears a sun cap, the cap body tightly fits the top of the head of the user. The visor is disposed at the front edge of the body. When the user is wearing the sun cap, the visor is positioned above the eyes of the user to shield the user from the sun so that the face, especially the eyes, of the user is not directly exposed to the sun.

There are two main types of sun cap bodies available in the marketplace. One type of sun cap bodies covers the head entirely, and the other type takes the form of a headband which fits around the head of a user. As shown in FIG. 1, the cap body in the form of a headband is generally a soft strip made of cloth. The rear side of the headband is disposed with a flexible adjusting means made of materials like Velcro, thereby allowing the user to adjust the size of the cap body according to the size of the head. When a user wears this kind of sun cap, as the cloth-made headband tightly fits the head for a long time, the user will easily swelter and feel uncomfortable, especially in hot weather. Moreover, cloth-made materials are not waterproof. The materials easily get wet when they are in contact with water and it takes time to clean and dry them. Therefore, they are not suitable for water activities.

Sun cap bodies in the form of headbands can be improved to take the form of visor arms which engage with only part of the head. As shown in FIG. 2, a sun cap with visor arms is generally made by plastic injection molding. The cap body and the visor are integrally formed as a whole. As the plastic body is elastic, the user simply slips the body around the head and the body will automatically fit the head. In comparison with a sun cap with headband, the contact area between a user's head and a sun cap with visor arms is smaller, thereby alleviating sweltering of the user while the user wears the sun cap. However, sun cap with visor arms are large and bulky, and they are made of harder materials. The sun cap tightly clasps the head of the user while in use, making the user feel uncomfortable and thus are not suitable for wearing for a long time. If a user wears a sun cap with visor arms in a larger size, the sun cap will easily fall off. It therefore has a low practicability and is difficult to be used in sports activities. In addition, sun caps with visor arms cannot be folded and therefore it is not easy to store them and carry them around.

### BRIEF SUMMARY OF THE INVENTION

In view of the aforesaid disadvantages now present in the prior art, the object of the present invention is to provide a simple sun visor which is comfortable to wear, convenient to take off and wear, small in size, waterproof and floatable on water, easy to wash, low in production cost, fashionable, easy to store and carry around.

To attain this, the present invention generally comprises a visor, and a left and a right elastic supporting arms which are in the shape of thin and curved strips. The left supporting arm engages with a left end of the visor. The right supporting arm engages with a right end of the visor. An outer surface of an upper portion of the visor is disposed with an elastic plastic pad having slip-proof and shockproof properties. The pad can be made of silica gel, rubber or other soft plastics like polyurethane (PU) for better fitting of the visor to user's forehead, thereby preventing displacement and falling off of the sun visor caused by user's activities, reducing vibration caused by strong air current striking against user's forehead and making the sun visor more comfortable to wear. The supporting arms can be made of plastic or metallic materials which are elastic and thus fit user's head better and make the sun visor more stable and secure to wear.

An upper edge of the visor is disposed with an elastic plastic rim having slip-proof and shockproof properties which is integrally formed with the pad as a whole.

An inner surface of the upper portion of the visor is disposed with an elastic plastic pad having slip-proof and shockproof properties.

The left and right supporting arms fixedly engage with the left and right ends of the visor respectively by means of screws.

The left and right supporting arms engage with the left and right ends of the visor respectively by means of hinge shafts. Each of the hinge shafts is disposed substantially vertically at the respective end of the visor. Each of the supporting arms pivots inward on the respective hinge shaft and closes at a rear side of the visor.

The left and right supporting arms engage with the left and right ends of the visor respectively by means of rotation shafts. Each of the rotation shafts is disposed substantially horizontally at the respective end of the visor. Each of the supporting arms pivots upward on the respective rotation shaft and crosses the upper edge of the visor to rest on a front side of the visor.

The left and right supporting arms are of foldable construction. Each of the left and right supporting arms comprises a shorter section and a longer section connected with each other by means of hinge shafts. Each of the shorter sections fixedly engages with the respective end of the visor. Each of the longer sections pivots inward on the hinge shaft and closes at the rear side of the visor to become folded.

The left and right supporting arms have sliding track structures. The front side or each of the two ends of the visor is disposed with a sliding track member. Each of the left and right supporting arms couples with the sliding track member and slides along the sliding track member to rest on the front side of the said visor or to extend from the ends of the visor for use. The sliding track member takes the form of sliding rails.

Each of the left and right supporting arms has an end wrapped up in or connected to a soft fixing piece. The soft fixing pieces engage with the inner sides of user's ears during wear to strengthen the attachment to the inner sides of the ears, thereby making it more secure to wear the present invention.

The visor can be made of materials like plastics, rubber or fibers. It can be made of lens having anti-ultraviolet ray or anti-fog properties which are shaped into certain curvature during manufacture. The visor has a thickness of 0.5-3 mm and a width of 30-80 mm at a widest portion thereof.

The visor can be of double-layered construction which comprises an upper layer visor and a lower layer visor. The upper and lower layer visors are attached to each other in a way which allows the lower layer visor to slide forward or backward relative to the upper layer visor so as to adjust the overall length of the visor.

The sun visor further comprises a sunglasses lens. Two ends of the sunglasses lens are movably engaged with the two ends of the visor respectively by means of hinge shafts.

The principle of operation of the present invention is similar to that of sunglasses. By means of the two supporting arms, part of the supporting force is focused on the inner sides of the two ears, thereby reducing excessive pressure caused by the sun visor clasping the head. In comparison with similar sun visors which clasp the head tightly, the present invention is more comfortable and reasonable. The present invention comprises supporting arms which are foldable, rotational or slidable. To use the present invention, unfold the supporting arms to form a supporting construction similar to that of the arms of eyeglasses. The user can then fit the supporting arms onto the inners sides of the two ears, and at the same time attach the inner side of the visor to the forehead. In this way, the present invention is worn to shield the user from the sun. After use, the user takes off the sun visor of the present invention. The supporting arms can be folded to the inner side or the outer side of the visor to greatly reduce the size of the present invention for easy portability or storage.

In comparison with the prior art, the present invention has the following advantages and effects:

(1) To overcome the disadvantage that sun caps with headband causes sweltering and uncomfortable feeling during wear, the present invention is adapted to have a smaller contact area between the supporting arms and the user's head. As a result, the user will not swelter while wearing the present invention. Furthermore, the present invention is lighter and more comfortable in comparison with existing sun caps.

(2) To overcome the disadvantage that the cloth-made headbands of sun caps are not waterproof, easy to get wet and time-consuming to clean, the supporting arms and the visor of the present invention are adapted to be made of waterproof materials. As a result, the present invention is easy to clean and quick to dry, making it an all-weather sports article which is particularly suitable for outdoor or even water activities.

(3) To overcome the disadvantage that the body of sun caps with visor arms are large and bulky, the weight of the supporting arms of the present invention is adapted to be relatively light. Moreover, as the present invention does not have a forehead portion but directly uses the inner edge of the upper portion of the visor as the forehead portion, the overall weight of the present invention is reduced. The present invention is thus lighter. Even if the user wears the present invention for a long time, the user will not feel uncomfortable. The materials for manufacturing the visor body are also greatly reduced, thereby lowering the production cost.

(4) To overcome the disadvantage that the materials of sun caps with visor arms are generally harder that make the user feel uncomfortable as the visor arms tightly clasp the user's during use, the supporting arms of the present invention are adapted to be made of plastic or metallic materials which are smaller in size and more elastic, thereby fitting the head of the user better and making the present invention more stable, comfortable and secure to wear. It also lessens the uncomfortable feeling caused by the pressure exerted by one-piece sun caps formed by injection molding which tightly clasp the head due to their rigid shape, bulkiness and low flexibility.

(5) To overcome the disadvantage that sun cap with visor arms cannot be folded for storage, the present invention is adapted to comprise supporting arms which are foldable, slidable or rotational. It is convenient to unfold or fold the present invention. It is also easy to store or carry around when the present invention is not in use. The present invention is very suitable for traveling, going outdoors or playing different kinds of sports (including water sports).

(6) The present invention is provided with an elastic plastic pad which is slip-proof and shockproof. The visor fits the forehead of the user better, thereby preventing displacement or falling off of the present invention caused by user's activities, reducing vibration caused by strong air current striking against the user's forehead and making the present invention more comfortable to wear.

(7) In comparison with the hat caps with headband, the double-arm construction of the present invention enables it to float on water due to mass-surface ratio as a result of the removal of extra burden caused by the forehead portion. Unlike sun caps which sink when submerged in water, the present invention is able to float when submerged in water. Especially to users involving in water activities, the present invention reduces the chances of losing the sun cap when the sun cap falls in the water.

(8) As the supporting arms and the visor of the present invention are two independent units, they can be manufactured independently into different styles or made of different materials according to the needs of the consumers and then assembled together. Even if the head circumference is changed, there is no need to develop a new mold. This greatly lowers the production cost, increases the adaptability and flexibility of product design and better meets the market demands.

(9) The design of the supporting arms of the present invention allows the user to wear and take off the present invention easily and conveniently without messing up the hair, making the user feel comfortable and relieved.

### BRIEF DESCRIPTION OF THE DRAWINGS

FIG. 1 shows the construction of an existing sun cap.

FIG. 2 shows the construction of another existing sun cap.

FIG. 3 shows the construction of the present invention.

FIG. 4 shows another construction of the present invention.

FIG. 5 shows the third construction of the present invention.

FIG. 6 shows the fourth construction of the present invention.

FIG. 7 shows the fifth construction of the present invention.

FIG. 8 shows the sixth construction of the present invention.

FIG. 9 shows the seventh construction of the present invention.

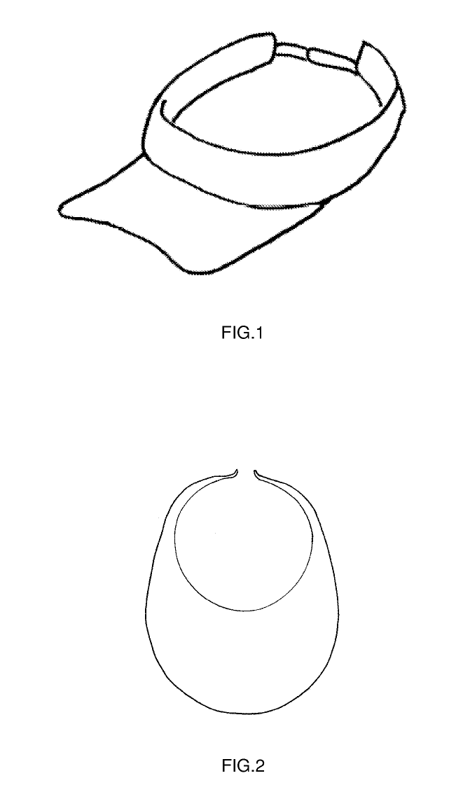

### DETAILED DESCRIPTION OF THE INVENTION

The preferred embodiments of the present invention are further described in detail with the following embodiments and the accompanying drawings. However, the present invention is capable of other embodiments.

### Embodiment 1

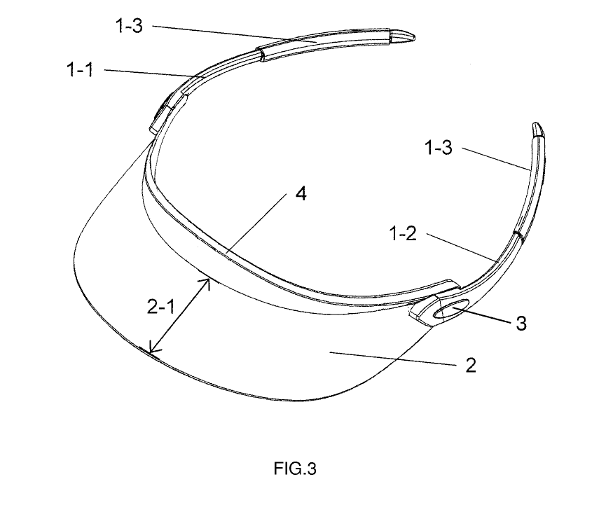

FIG. 3 illustrates the construction of the present invention. As shown in FIG. 3, the present invention comprises a visor 2, and a left and a right elastic supporting arms 1-1, 1-2 which are in the shape of thin and curved strips. The left and right supporting arms 1-1, 1-2 fixedly engage with the two ends of the visor 2 respectively. The engagement is achieved by means of screws 3. In other embodiments, the engagement can also be achieved by other clipping means. The supporting arms 1-1, 1-2 can be made of metallic materials or plastics which form certain curvature after formation and can fit the user's head better. Each of the left and right supporting arms 1-1, 1-2 has an end wrapped up in a soft fixing pieces 1-3. In other embodiments, the ends of the supporting arms can also connect to plug-in type soft fixing pieces (not shown). The plug-in type soft fixing pieces can be independently replaced by fixing pieces of different lengths or hardness so as to fit different head circumferences and cater for different people's preference on the hardness of the soft fixing pieces. Better flexibility is thereby achieved. In addition, the ends of the left and right supporting arms 1-1, 1-2 can also be disposed with no soft fixing pieces. Instead, the supporting arms can be made of plastic or rubber materials which can better attached to human skins. The supporting arms can therefore be directly and tightly attached to the inner sides of user's ears. It is more secure to use, relatively simple in construction and easier to manufacture. When the user wears the present invention, the soft fixing pieces 1-3 engage with the inner sides of user's ears to strengthen the attachment to the inner sides of the ears, thereby making it more secure to wear the present invention. In this embodiment, an outer surface of the visor 2 is disposed with an elastic plastic pad having slip-proof and shockproof properties. The pad is made of silica gel. In other embodiments, the pad can also be made of rubber or other soft plastics like polyurethane (PU) for better fitting of the visor 2 to user's forehead, thereby preventing displacement and falling off of the present invention caused by user's activities, reducing vibration caused by strong air current striking against user's forehead and making the present invention more comfortable to wear. In this embodiment, an upper edge of the visor 2 is disposed with an elastic plastic rim 4 having slip-proof and shockproof properties which is integrally formed with the pad as a whole. However, in other embodiments, to simplify the manufacturing process and to lower the production costs, the visor 2 can be disposed only with the pad but without the rim 4. Moreover, in other embodiments, an inner surface of the visor 2 can also be disposed with an elastic plastic pad having slip-proof and shockproof properties. The visor 2 can be made of a common plastic sheet which is shaped into certain curvature during manufacture. The visor has a thickness of 0.5-3 mm and a width of 30-80 mm at a widest portion thereof. In other embodiments, the visor 2 can also be made of materials like rubber, fibers and plastics. The visor can be a lens having anti-ultraviolet ray or anti-fog properties.

To use the present invention, the user can fit the supporting arms 1-1, 1-2 onto the inner sides of the two ears, and at the same time attach the inner side of the visor 2 to the forehead. In this way, the present invention is worn to shield the user from the sun.

### Embodiment 2

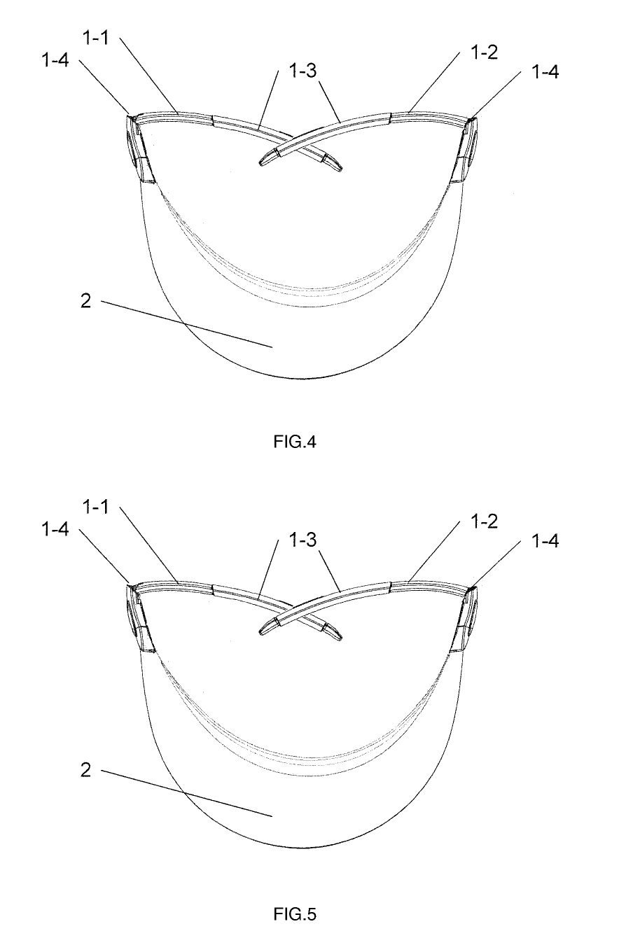

FIG. 4 illustrates another construction of the present invention. As shown in FIG. 4, this embodiment is the same as embodiment 1 except for the following characteristics. In this embodiment, the left and right supporting arms 1-1, 1-2 are of foldable construction. Each of the left and right supporting arms 1-1, 1-2 comprises two sections connected with each other by means of hinge shafts 1-4. Each of the shorter sections fixedly engages with the respective end of the visor 2. Each of the longer sections pivots inward on the hinge shaft 1-4 and closes at the rear side of the visor 2 to become folded. To use the present invention, unfold the supporting arms 1-1, 1-2 to form a supporting construction similar to that of the arms of eyeglasses. The user can then fit the supporting arms 1-1, 1-2 onto the inner sides of the two ears, and at the same time attach the inner side of the visor 2 to the forehead of the user. In this way, the present invention is worn to shield the user from the sun. After use, the user takes off the present invention. The supporting arms 1-1, 1-2 can be folded to the rear side of the visor 2 to greatly reduce the size of the present invention for easy portability or storage.

### Embodiment 3

FIG. 5 illustrates the third construction of the present invention. As shown in FIG. 5, this embodiment is the same as embodiment 1 except for the following characteristics. In this embodiment, the left and right supporting arms 1-1, 1-2 have sliding track structures. The front side of the visor 2 is disposed with a sliding track member. Each of the left and right supporting arms 1-1, 1-2 couples with the sliding tracks member and slides along the sliding tracks member to rest on the front side of the visor 2 or extend from the ends of the visor 2 for use.

### Embodiment 4

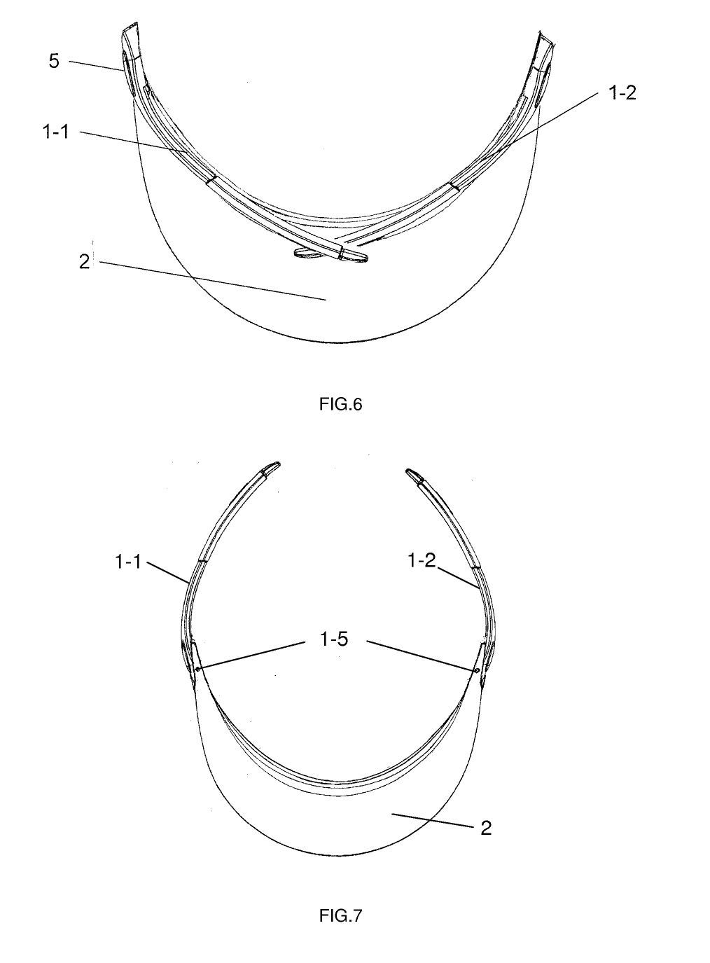

FIG. 6 illustrates the fourth construction of the present invention. As shown in FIG. 6, this embodiment is the same as embodiment 1 except for the following characteristics. In this embodiment, the left and right supporting arms 1-1, 1-2 engage with the two ends of the visor 2 respectively by means of rotation shafts 5. Each of the rotation shafts 5 is disposed substantially horizontally at the respective end of the visor 2. Each of the supporting arms pivots upward on the rotation shafts 5 and crosses the upper edge of the visor 2 to rest on the front side of the visor 2.

### Embodiment 5

FIG. 7 illustrates the fifth construction of the present invention. As shown in FIG. 7, this embodiment is the same as embodiment 1 except for the following characteristics. In this embodiment, the supporting arms 1-1, 1-2 engage with the two ends of the visor 2 respectively by means of hinge shafts 1-5. Each of the hinge shafts 1-5 is disposed substantially vertically at the respective end of the visor 2. Each of the supporting arms 1-1, 1-2 pivots inward on the respective hinge shaft 1-5 and closes at the rear side of the visor 2.

### Embodiment 6

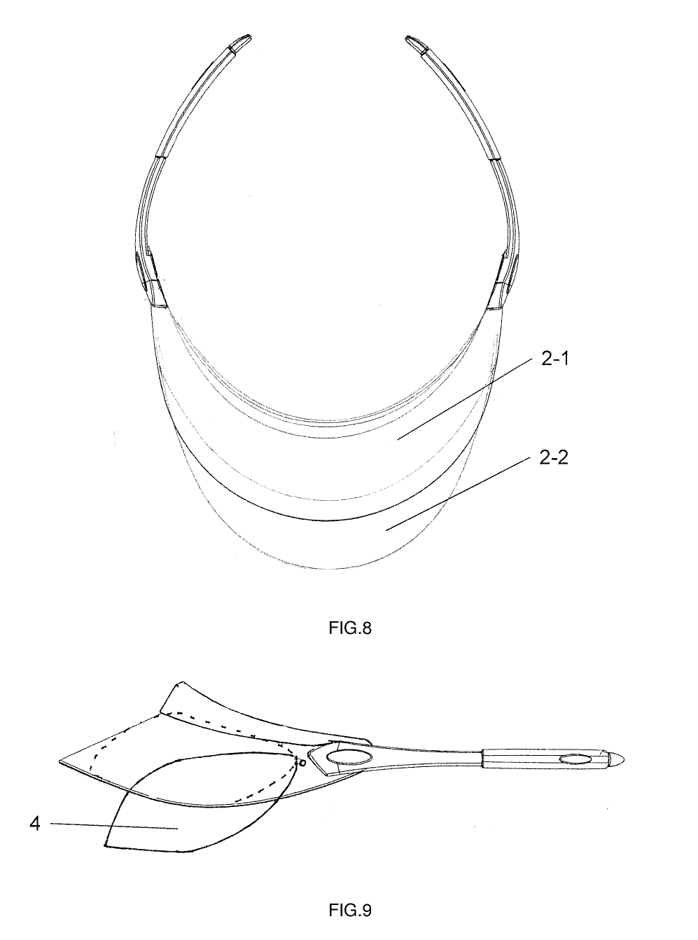

FIG. 8 illustrates the sixth construction of the present invention. As shown in FIG. 8, this embodiment is the same as embodiment 1 except for the following characteristics. In this embodiment, the visor 2 is of double-layered construction which comprises an upper layer visor 2-1 and a lower layer visor 2-2. The upper and lower layer visors 2-1, 2-2 are attached to each other in a way which allows the lower layer visor 2-2 to slide forward or backward relative to the upper layer visor 2-1 so as to adjust the overall length of the visor 2.

### Embodiment 7

FIG. 9 illustrates the seventh construction of the present invention. As shown in FIG. 9, this embodiment is the same as embodiment 1 except for the following characteristics. This embodiment is disposed with an extra removable sunglasses lens 4. The sunglasses lens 4 provides UV 400 protection. When the eyes are exposed to a large amount of ultraviolet light, the user can move the sunglasses lens 4 downward to the front of the eyes to further prevent the strong ultraviolet light from penetrating into the eyes. When the sunlight weakens, the user can return (take back) the sunglasses lens 4 to its original place. This can prevent the pupils of the eyes from dilating for a long time due to seeing through dark lenses for a long time and losing the self-protection mechanism which contrarily increasing the chance of the eyes being penetrated and hurt by the ultraviolet light. Two ends of the sunglasses lens 4 are engaged with the two ends of the visor respectively by means of hinge shafts or other means.

The above embodiments are preferred embodiments of the present invention. The present invention is capable of other embodiments and is not limited by the above embodiments. Any other variation, decoration, substitution, combination or simplification, whether in substance or in principle, not deviated from the spirit of the present invention, is replacement or substitution of equivalent effect and falls within the scope of protection of the present invention.

What is claimed is:

1. A simple sun visor, wherein it comprises a visor, and a left and a right elastic supporting arms which are in the shape of thin and curved strips; the left supporting arm engages with a left end of the visor; the right supporting arm engages with a right end of the visor; an outer surface of an upper portion of the visor is disposed with an elastic plastic pad having slip-proof and shockproof properties.

2. The simple sun visor as in claim 1, wherein an upper edge of the visor is disposed with an elastic plastic rim having slip-proof and shockproof properties which is integrally formed with the pad as a whole.

3. The simple sun visor as in claim 1, wherein an inner surface of the upper portion of the visor is disposed with an elastic plastic pad having slip-proof and shockproof properties.

4. The simple sun visor as in claim 1, wherein the left and right supporting arms engage with the left and right ends of the visor respectively by means of hinge shafts; each of the hinge shafts is disposed substantially vertically at the respective end of the visor; each of the supporting arms pivots inward on the respective hinge shaft and closes at a rear side of the visor.

5. The simple sun visor as in claim 1, wherein the left and right supporting arms engage with the left and right ends of the visor respectively by means of rotation shafts; each of the rotation shafts is disposed substantially horizontally at the respective end of the visor; each of the supporting arms pivots upward on the respective rotation shaft and crosses the upper edge of the visor to rest on a front side of the visor.

6. The simple sun visor as in claim 1, wherein the left and right supporting arms are of foldable construction; each of the left and right supporting arms comprises a shorter section and a longer section connected with each other by means of hinge shafts; each of the shorter sections fixedly engages with the respective end of the visor; each of the longer sections pivots inward on the hinge shaft and closes at the rear side of the said visor to become folded.

7. The simple sun visor as in claim 1, wherein the left and right supporting arms have sliding track structures; the front side or each of the two ends of the visor is disposed with a sliding track member; each of the left and right supporting arms couples with the sliding track member and slides along the sliding track member to rest on the front side of the visor or to extend from the ends of the visor for use.

8. The simple sun hat as in claim 1, wherein each of the left and right supporting arms has an end wrapped up in or connected to a soft fixing piece; the soft fixing pieces engage with inner sides of user's ears to strengthen the attachment to the inner sides of the ears, thereby making it more secure to wear the present invention.

9. The simple sun visor as in claim 1, wherein the visor can be of double-layered construction which comprises an upper layer visor and a lower layer visor; the upper and lower layer visors are attached to each other in a way which allows the lower layer visor to slide forward or backward relative to the upper layer visor so as to adjust the overall length of the visor.

10. The simple sun visor as in claim 1, wherein the said sun visor further comprises a sunglasses lens; two ends of the sunglasses lens are movably engaged with the two ends of the visor respectively by means of hinge shafts.
